(19) United States
(12) Patent Application Publication
(10) Pub. No.: US 2009/0024148 A1
(43) Pub. Date: Jan. 22, 2009

(54) METHOD FOR FORMING PLICATIONS OF THE GASTRIC CAVITY

(76) Inventors: Mark S. Zeiner, Mason, OH (US); Michael J. Stokes, Cincinnati, OH (US); Daniel E. Alesi, Lebanon, OH (US); Jason L. Harris, Mason, OH (US); Lawrence Crainich, Charlestown, NH (US)

Correspondence Address: WELSH & FLAXMAN LLC, 2000 DUKE STREET, SUITE 100, ALEXANDRIA, VA, 22314, US

(21) Appl. No.: 12/113,836

(22) Filed: May 1, 2008

Related U.S. Application Data
(63) Continuation-in-part of application No. 11/779,322, filed on Jul. 18, 2007.

(51) Int. Cl. A61B 17/08

(52) U.S. Cl. 606/151

(57) ABSTRACT

A method for forming plications of the gastric cavity is achieved by forming a tissue fold along a gastric wall of the gastric cavity and securing the tissue fold with a fastener, wherein the step of securing includes positioning at least one buttress between the tissue of the gastric wall and the fastener.

### CROSS REFERENCE TO RELATED APPLICATIONS

This application is continuation-in-part of U.S. patent application Ser. No. 11/779,322, entitled “HYBRID ENDOSCOPIC/LAPAROSCOPIC METHOD FOR FORMING SEROSA TO SEROSA PLICATIONS IN A GASTRIC CAVITY”, filed Jul. 18, 2007, which is currently pending.

### BACKGROUND OF THE INVENTION

1. Field of the Invention

The invention relates to gastric reduction surgery. More particularly, the invention relates to the deployment and distribution of load in the application of fasteners during gastric reduction surgery.

2. Description of the Related Art

Obesity is a medical condition affecting more than 30% of the population in the United States. Obesity affects an individual's personal quality of life and contributes significantly to morbidity and mortality. Obese patients, i.e., individuals having a body mass index (“BMI”) greater than 30, often have a high risk of associated health problems (e.g., diabetes, hypertension and respiratory insufficiency), including early death. With this in mind, and as those skilled in the art will certainly appreciate, the monetary and physical costs associated with obesity are substantial. In fact, it is estimated the costs relating to obesity are in excess of 100 billion dollars in the United States alone. Studies have shown that conservative treatment with diet and exercise alone may be ineffective for reducing excess body weight in many patients. Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In a RYGB procedure a small stomach pouch is separated from the remainder of the gastric cavity and attached to a resectioned portion of the small intestine. This resectioned portion of the small intestine is connected between the “smaller” gastric cavity and a distal section of small intestine allowing the passage of food therebetween. The conventional RYGB procedure requires a great deal of operative time. Because of the degree of invasiveness, post-operative recovery can be quite lengthy and painful. Still more than 100,000 RYGB procedures are performed annually in the United States alone, costing significant health care dollars.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing a feeling of satiety. A band is placed around the stomach near the junction of the stomach and esophagus. The small upper stomach pouch is filled quickly, and slowly empties through the narrow outlet to produce the feeling of satiety. In addition to surgical complications, patients undergoing a gastric banding procedure may suffer from esophageal injury, spleen injury, band slippage, reservoir deflation/leak, and persistent vomiting. Other forms of bariatric surgery that have been developed to treat obesity include Fobi pouch, bilio-pancreatic diversion and gastroplasty or “stomach stapling”.

Morbid obesity is defined as being greater than 100 pounds over one's ideal body weight. For individuals in this category, RYGB, gastric banding or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss.

Various mechanisms have been developed for reconfiguring the stomach as part of a weight loss program. However, it is difficult to reconfigure the stomach to promote weight loss for an extended amount of time. Ultimately, the stomach will organize itself into its original shape. Fasteners have historically eroded through the gastric wall, that is, suture, t-tags, staples, etc. Also, through preclinical experiments, it has been determined that a serosa-to-serosa connection is more durable than a mucosa-to-mucosa connection.

With the foregoing in mind, it is desirable to have a surgical weight loss procedure that is inexpensive, with few potential complications, and that provides patients with a weight loss benefit while buying time for the lifestyle changes necessary to maintain the weight loss. Further, it is desirable that the procedure be minimally invasive to the patient, allowing for a quick recovery and less scarring. The present invention provides such a procedure.

### SUMMARY OF THE INVENTION

It is, therefore, an object of the present invention to provide a method for forming plications of the gastric cavity. The method is achieved by forming a tissue fold along a gastric wall of the gastric cavity and securing the tissue fold with a fastener, wherein the step of securing includes positioning at least one buttress between the tissue of the gastric wall and the fastener.

It is also an object of the present invention to provide a method wherein the fastener is a t-tag fastener.

It is another object of the present invention to provide a method wherein the tissue fold is formed in an anterior wall of the gastric cavity.

It is a further object of the present invention to provide a method wherein the tissue fold is a serosa-to-serosa fold.

It is also an object of the present invention to provide a method wherein the at least one buttress is annular shaped.

It is another object of the present invention to provide a method wherein the buttress is an elongated member including a plurality of apertures through which a plurality of fasteners are respectively applied in a manner holding the tissue fold together.

It is a further object of the present invention to provide a method including the step of deploying multiple buttresses within the gastric cavity, wherein the step of deploying includes inserting a plurality of buttresses over an endoscopic grasper and opening grasper jaws of the endoscopic grasper to prevent the plurality of buttresses from falling off the endoscopic grasper, transorally delivering the endoscopic grasper to the gastric cavity and closing the grasper jaws of the endoscopic grasper to release the buttress within the gastric cavity.

It is also an object of the present invention to provide a method including the step of deploying multiple buttresses within the gastric cavity, wherein the step of deploying includes aligning a series of buttresses along a longitudinal axis and wrapping a suture therearound to hold the buttresses together, engaging the buttresses with an endoscopic grasper, delivering the buttresses into the gastric cavity, and releasing the buttresses to fall off inside the gastric cavity.

It is another object of the present invention to provide a method including the step of deploying multiple buttresses within the gastric cavity, wherein the step of deploying includes providing a series of buttresses, which are connected via fracture zones allowing selective separation thereof, engaging the series of buttresses with an endoscopic grasper, delivering the buttresses transorally into the gastric cavity, manipulating the buttresses until the fracture line between adjacent buttresses is broken at which time the buttress may be utilized at the surgical site in a desired manner.

It is a further object of the present invention to provide a method including the step of deploying multiple buttresses within the gastric cavity, wherein the step of deploying includes delivering each buttress with a loop of suture, wherein each loop of suture is tied to the next loop of suture such that consecutive buttresses are available as needed, releasing individual buttresses by cutting the suture loop releasing a buttress for use.

It is also an object of the present invention to provide a method including the step of deploying multiple buttresses within the gastric cavity, wherein the step of deploying includes transorally positioning a delivery device within the gastric cavity, the delivery device includes a housing in which a plurality of buttresses are stacked for subsequent dispensing and a dispensing aperture for selective release of buttresses held within the housing, wherein movement of the buttresses toward the dispensing aperture is achieved by the utilization of a push rod.

It is also an object of the present invention to provide a method including the step of deploying multiple buttresses within the gastric cavity, wherein the step of deploying includes supporting a plurality of buttresses upon a rack, wherein the rack includes support members for selective engagement of a series of buttresses which are aligned along a longitudinal axis such that central apertures of the buttresses are in controlled alignment for release within the gastric cavity.

It is still a further object of the present invention to provide a method including the step of deploying multiple buttresses within the gastric cavity, wherein the step of deploying includes supporting the buttresses within a housing wherein the buttresses include interlocking hooks that allow them to be maintained in an aligned arrangement within the housing, forcing the buttresses toward a dispensing aperture of the housing where a single buttress is exposed and released from engagement with the adjacent buttresses.

It is another object of the present invention to provide a method wherein the tissue fold is a serosa-to-serosa fold.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic view showing transoral access to the gastric cavity.

FIGS. 2-6 are various views showing the steps associated with the formation of a serosa-to-serosa fold along the anterior wall of the gastric cavity.

FIG. 7 is a perspective view showing use of a buttress in the formation of a serosa-to-serosa fold.

FIGS. 8 and 9 respectively show a perspective view and cross sectional view of the buttress of FIG. 7 being used in accordance with the present invention.

FIG. 10 is a perspective view showing a buttress in accordance with an alternate embodiment in the formation of a serosa-to-serosa fold.

FIG. 11 is a side view of a delivery mechanism utilized in deploying a plurality of buttresses within the gastric cavity.

FIG. 12 shows an alternate deployment mechanism for utilization in deploying multiple buttresses within the gastric cavity.

FIG. 13 shows a series of buttresses connected by fracture zones for deployment within the gastric cavity and subsequent individual use in accordance with the present invention.

FIG. 14 is a side view showing an alternate deployment mechanism for utilization in accordance with the present invention.

FIGS. 15 and 16 are respectively a perspective view and a side view of a buttress delivery device for utilization in accordance with the present invention.

FIG. 17 discloses a rack mechanism for deployment of buttresses in accordance with the present invention.

FIG. 18 is yet another deployment mechanism for utilization in deploying buttresses within the gastric cavity for use in the formation of a serosa-to-serosa fold.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

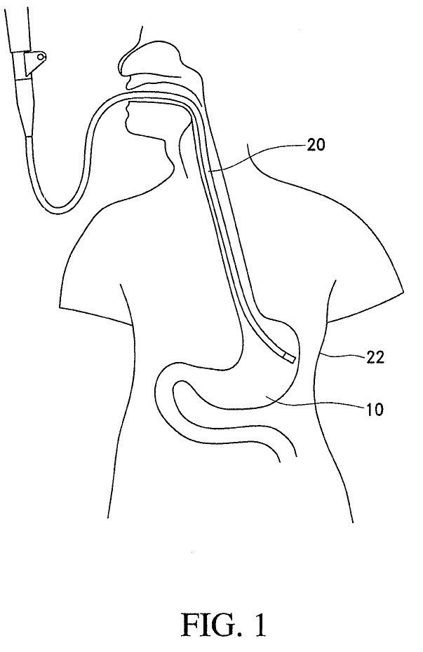

With regard to the various embodiments disclosed herein, and with reference to FIGS. 1 to 6, a mechanism for creating a serosa-to-serosa fold to reduce the volume of the gastric cavity 10 using a suture anchoring device, for example, a t-tag fastener, 12 is disclosed. The present procedure employs one or more buttresses 14 positioned between the tissue of the gastric wall 16 and the t-tag fastener 12 to enhance the fold created thereby. The buttress methodology disclosed in accordance with the present invention distributes the load from the t-tag fastener 12 onto the tissue in order to limit or reduce erosion through the gastric wall 16.

In accordance with a preferred embodiment of the present invention, available stomach volume may be restricted by forming one or more folds in the anterior wall 16 of the gastric cavity 10 (creating a serosa-to-serosa fold 18). The folds 18 reduce the outer surface area of the gastric cavity 10 and, correspondingly, the available food volume within the gastric cavity 10. In accordance with one restriction technique, the available volume within the gastric cavity 10 is restricted by forming a single, longitudinally extending fold 18 along the anterior wall 16 of the gastric cavity 10. The fold 18 extends the full length of the anterior wall 16 of the gastric cavity 10 between the fundus and the pylorus. Alternatively, a shorter fold may be formed depending upon the desired amount of gastric volume reduction.

Generally, to form a fold in accordance with the present invention, a flexible gastroscope 20 is passed transesophageally into the gastric cavity 10 as shown in FIG. 1. The gastroscope 20 provides insufflation, illumination, and visualization of the gastric cavity 10, as well as a passageway into the gastric cavity 10 for the insertion and use of other endoscopic instruments. The gastric cavity 10 is first insufflated to create a sufficient rigid working surface along the gastric cavity 10 such that it may be pierced without damaging the opposing wall of the gastric cavity 10. Insufflation of the gastric cavity 10 may also allow the boundaries of the gastric cavity 10 and the desired location for a fold 18 to be mapped out by external palpation. The pressure on the abdominal wall 22 is observed within the gastric cavity 10 through the gastroscope 20 to also determine the appropriate placement of one or more trocars (or other ports allowing abdominal access) for completion of the procedure in accordance with the present invention.

After the gastric cavity 10 has been mapped through the gastroscope 20, a trocar 24 is inserted through the abdominal wall 22 and then the gastric wall 10. FIG. 2 shows a trocar 24 inserted through the abdominal wall 22 and directly above the gastric cavity 10. The placement of the trocar 24 depends upon the intended location of the fold 18, in particular, the serosa-to-serosa fold. It should be noted that with insuflation of the peritoneal cavity the trocar could be inserted in the same locations as are typically used for gastric banding or RYGB procedures (that is, not directly above the stomach). The trocar 24 preferably has a diameter of between approximately 3 mm and approximately 5 mm to allow an adequate sized passageway for instruments and suture anchoring devices, that is, t-tag fasteners in accordance with a preferred embodiment of the present invention, 12 employed in accordance with the implementation of the techniques described herein.

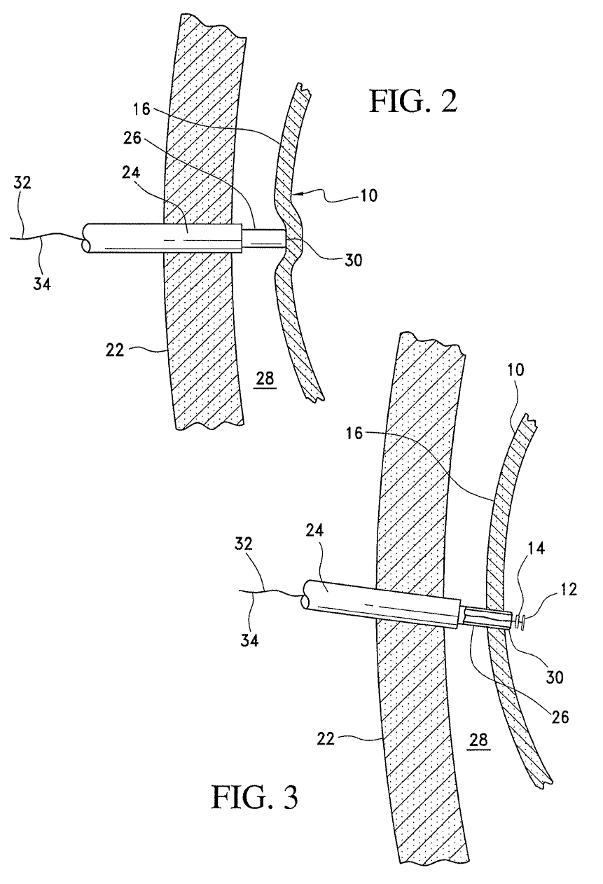

With the trocar inserted into the abdominal wall 22, a suture anchor deployment device 26 is passed through the trocar 24 into the abdominal cavity 28. Prior to insertion of the deployment device 26, the tip 30 of the deployment device 26 is pressed against the anterior wall 16 of the gastric cavity 10 to indent the wall, as shown in FIG. 2. The indentation along the anterior wall 16 of the gastric cavity 10 is visualized through the gastroscope 20 (from within the stomach) to determine the proper location to insert the deployment device 26 into the gastric cavity 10. After the proper insertion location is determined, the tip 30 of the deployment device 26 is inserted through the anterior wall 16 and into the interior of the gastric cavity 10. The deployment device 26 is inserted into the gastric cavity 10 with sufficient force to prevent the deployment device 26 from glancing off of the exterior surface of the anterior wall 16 of the gastric cavity 10. After the tip 30 of the deployment device 26 is inside the gastric cavity 10, as shown in FIG. 3, a t-tag fastener 12 is deployed from the deployment device 26 into the interior of the gastric cavity 10 and the t-tag fastener 12 engages the buttress 14 (which was previously positioned within the gastric cavity 10 as discussed below in greater detail) with the suture material 32 from the t-tag fastener 12 extending through a central aperture 40 of the buttress 14. While a single t-tag fastener and buttress are shown in accordance with this embodiment, the following disclosure will show that multiple t-tag fasteners and buttresses may be employed within the spirit of the present invention.

While t-tag fasteners are disclosed for use in accordance with a preferred embodiment of the present invention, other suture anchoring devices may be utilized within the spirit of the present invention. Examples of suitable tissue fasteners include t-type anchors as already discussed, reconfigurable “basket”-type anchors (which generally comprise a number of configurable structure legs extending between at two collars or support members), and linear anchors (elongated anchors which are configured to fold or become compressed in to a bowed or expanded configuration). In general, anchor characteristics are such that prior to deployment, they can easily be placed into or through tissue(s), but after deployment, have an altered configuration providing at least one dimension sufficiently large to maintain the anchor in place. The specific structure of the buttress may take a variety of forms as discussed below in accordance with the various embodiments making up the present invention.

After the t-tag fastener 12 and buttress 14 are deployed into the gastric cavity 10, the deployment device 26 is removed from the gastric cavity 10. As the deployment device 26 is removed, the suture material 32 attached at the distal end to the t-tag fastener 12 extends from the t-tag fastener 12 and through the anterior wall 16 of the gastric cavity 10. The proximal end 34 of the suture material 32 extends through the trocar 24 and outside the body.

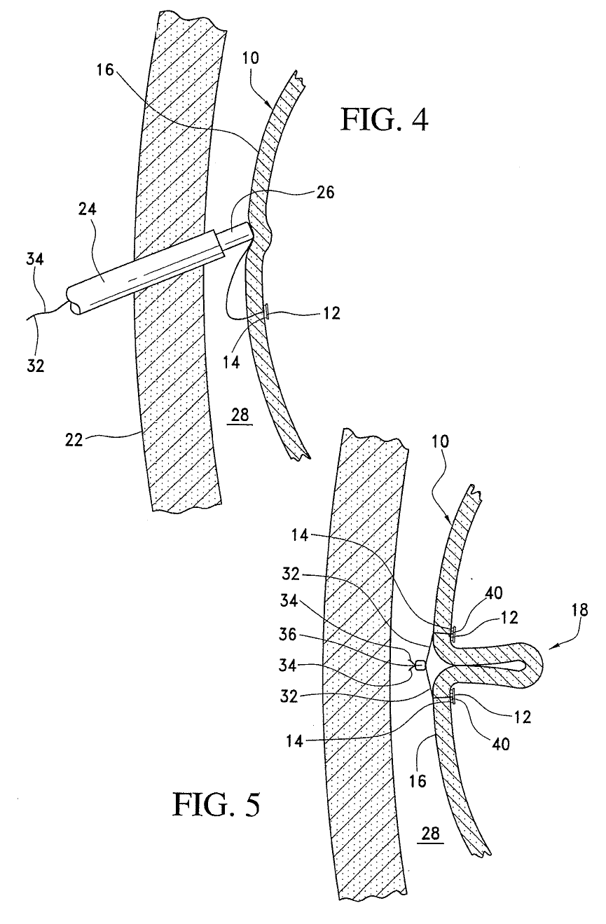

After the deployment device 26 is removed from the anterior wall 16 of the gastric cavity 10, the anterior wall 16 again is probed with the tip 30 of the deployment device 26 to determine the location for a second t-tag fastener 12 and buttress 14. To facilitate the probing of the anterior wall 16, the trocar 24 may be flexed at different angles within the abdominal wall 22 as shown in FIG. 4 without removing the trocar 24 from the abdominal wall 22. The trocar 24 is angled within the abdominal wall 22 to enable the deployment device 26 to enter the gastric cavity 10 at different locations and in a different direction roughly perpendicular to the exterior surface of the gastric cavity 10. If the angles are such that, perpendicular to the exterior surface is not achievable then a grasper can be used through another trocar port to bring the tissue up to allow a perpendicular angle to the tissue. Once the proper placement location is determined, the deployment device 26 is once again inserted into the gastric cavity 10. With the deployment device 26 inside the gastric cavity 10, a second t-tag fastener 12 is deployed into the interior of the gastric cavity 10 and the t-tag fastener 12 engages the buttress 14 (which was previously positioned within the gastric cavity 10 as discussed below in greater detail) with the suture material 32 from the t-tag fastener 12 extending through a central aperture 40 of the buttress 14. A second length of suture material 32 is attached at a distal end of the second t-tag fastener 12. After the second t-tag fastener 12 and buttress 14 are deployed, the deployment device 26 is removed from the gastric cavity 10, drawing the length of suture material 32 back through the anterior wall 16 of the gastric cavity 10. The proximal ends 34 of the first and second lengths of suture material 32 are drawn through the trocar 24 and external of the body. Tension is then applied to the proximal ends 34 of the respective first and second lengths of suture material 32 to draw the fastened portion of the anterior wall 16 of the gastric cavity 10 together to form a serosa-to-serosa fold 18 as shown in FIG. 5. The first and second lengths of suture material 32 are then locked in a tensioned state by applying a knotted element 36 to the proximal ends 34 of the respective first and second lengths of the suture material 32. The knotting element 36 is passed back through the trocar 24 to a location between the abdominal wall 22 and the anterior wall 16 of the gastric cavity 10.

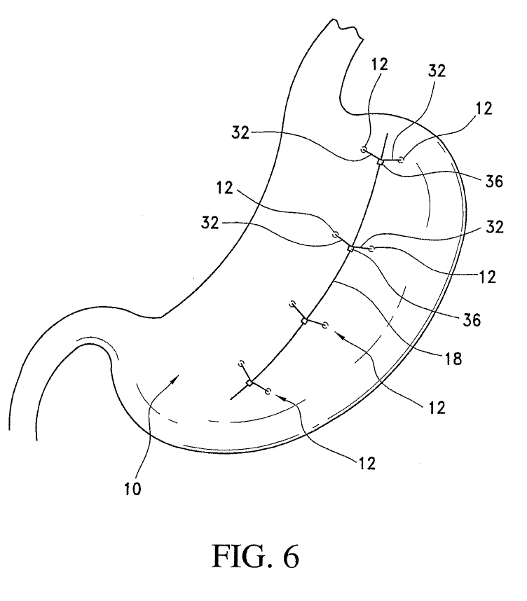

In addition to knotting elements, the suture material may also be locked in a tensioned state by tying a knot in the suture material. The knot may be tied laparoscopically through the trocar. Alternatively, the knot may be tied external of the body, and the finished knot passes back through the trocar to a point between the abdominal wall and the anterior wall of the gastric cavity. In an alternate embodiment, the first and second lengths of suture materials are pre-tied within the deployment device. The suture material may be of a sufficient length that the knot can be externalized from the body through the trocar, or can be short enough that laparoscopic manipulation is required to apply tension between the suture anchoring devices. In yet another embodiment, the t-tag fasteners are connected by a single piece of suture material (not shown) within the length chosen to be easily externalized or short enough to be completely tensioned internally. In either case, the suture material and t-tag fasteners may be pre-loaded within a deployment device in one or more sets, or can be loaded into cartridges that can be reloaded as needed. FIG. 6 shows an external view of the gastric cavity 10 with the t-tag fasteners 12 and the suture material 32 cinched between the t-tag fasteners 12 to form a serosa-to-serosa fold 18 therebetween. The knotting element 36 is shown applied to the suture material 32 to lock the tension in the suture material 32.

After the first pair of t-tag fasteners 12 and buttresses 14 are deployed, the trocar 24 and the deployment device 26 may be angled within the abdominal wall 22 to again probe the gastric cavity 10 and determine a third location for a t-tag fastener 12 and buttress 14. The third t-tag fastener 12 and buttress 14 are preferably spaced down the length of the anterior wall 16 from the first pair of t-tag fastener(s) 12 and buttress(es) 14, in order to extend the length of the fold 18. Once the third t-tag fastener 12 and buttress 14 location is determined, the deployment device 26 is again inserted through the anterior wall 16 of the gastric cavity 10 to deploy a third t-tag fastener 12 into the gastric cavity 10 where it is engaged with the buttress 14 as described above with regard to the first and second t-tag fasteners 12. Following deployment, the deployment device 26 is removed from the gastric cavity 10, and a fourth location is determined for placement of a t-tag fastener 12 and buttress 14. The t-tag fastener 12 and buttress 14 are spaced from the third t-tag fastener 12 and buttress 14 across the fold line. The deployment device 26 is inserted into the gastric cavity 10 at the fourth location, and a fourth t-tag fastener 12 is deployed into the gastric cavity 10, as shown in FIG. 6, where it is engaged with the buttress 14 as described above with regard to the first and second t-tag fasteners 12. The third and fourth lengths of suture material 32 extend from the third and fourth t-tag fasteners 12 through the anterior wall 14 of the gastric cavity 10. The third and fourth lengths of suture material 32 are cinched between the t-tag fasteners 12 and buttresses 14, and the suture tension locked in with a knotting element 36, to extend the fold 18. The third and fourth t-tag fasteners 12 and buttresses 14 are aligned longitudinally with the first and second t-tag fasteners 12 and buttresses 14, along the length of the anterior wall 16, so that a uniform wall fold 18 is formed between the pairs of t-tag fasteners 12 and buttresses 14.

As shown in FIG. 6, additional pairs of t-tag fasteners and buttresses may be positioned along the longitudinal length of the anterior wall of the gastric cavity. The trocar may be flexed within the abdominal wall, or removed and repositioned within the abdominal wall as necessary, in order to reach all of the desired t-tag fastener and buttress locations. The suture material is cinched together between each pair of the suture anchoring devices to extend the cavity wall fold. The number of t-tag fastener and buttress pairs used to form a fold will depend upon the desired length for the fold. (and also the depth of the fold or number of consecutive rows of fasteners) Preferably, each of the pairs of the t-tag fasteners and buttresses is evenly spaced apart along the length of the anterior wall of the gastric cavity. Likewise, within each individual pair the t-tag fasteners and buttresses are evenly spaced apart across the fold line, so that a uniform tissue is formed without distortion or bunching. The proper relative spacing of the t-tag fasteners and buttresses can be ascertained through the gastroscope. Alternatively, an additional trocar may be inserted into the abdominal wall and used in conjunction with an optical instrument to visually determine the proper locations for the t-tag fasteners and buttresses, as well as for the insertion of an additional grasper as discussed herein.

As an alternative to the embodiment described above, t-tag fasteners and/or buttresses may be passed through the gastroscope into the gastric cavity. An instrument may be passed on the end of or through the gastroscope for attaching the t-tag fastener(s) and buttress(es) into one of the gastric cavity walls to form a fold. Suture material may be tensioned adjacent to or through the gastroscope, and a knotting element passed adjacent to or through the gastroscope to the fold to lock in the suture tension.

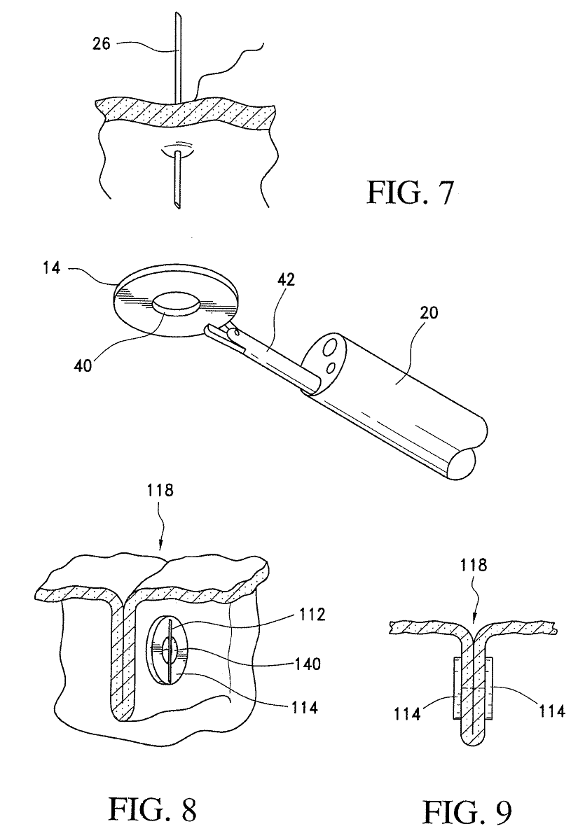

In general, and in accordance with a preferred embodiment of the present invention as shown with reference to FIG. 7 as well as preceding FIGS. 1 to 6, the methodology contemplated in accordance with the present invention requires internal buttresses 14 (for example, in the shape of a washer, that is, annular shaped) in the formation of a serosa-to-serosa fold 18. With regard to the internal buttresses 14 employed in accordance with a preferred embodiment of the present invention, they are composed of a biocompatible material and may take a variety of shapes as discussed below. The internal buttresses 14 are delivered into the gastric cavity 10 by use of an endoscopic grasper 42 as shown in FIG. 7. However, and as will be appreciated based upon the following disclosure, other delivery mechanisms, for example, a conveyor, pull string, pushing of a stack, cartridge on an endoscope, etc. may be employed. Once the internal buttresses 14 are in position, a t-tag fastener 12, or other suture anchor device, is placed through the central aperture 40 of the internal buttress 14 by the deployment device 26 and an endoscopic grasper 42. The grasper 42 positions the internal buttress 14 and the deployment device 26 positions the t-tag fastener 12 through the central aperture 40 of the internal buttress 14.

In accordance with a preferred embodiment, and as discussed below in greater detail, it is contemplated the internal buttresses may be stacked axially, linearly, etc. In addition, the internal buttresses may be independent and annular shaped like a conventional mechanical washer (or what is referred to below as an elongated buttress), connected with a fracture zone, or may be interconnected to form continuous bar. In addition, the internal buttresses may be injected into the tissue to help retain the t-tag fastener from migrating through the gastric wall. The incorporation of such internal buttresses may be achieved either before or after the t-tag fastener has been placed. In addition, various materials may be injected into the t-tag fastener site in order to promote toughness of the tissue to decrease erosion. Such materials may include schlerosants, tgf-beta, keratin, PMMA, etc.

More particularly, and with reference to FIGS. 8 and 9, a preferred serosa-to-serosa fold 118 is shown. In accordance with such an embodiment, a serosa-to-serosa fold 118 is created as described above. Internal buttresses 114 are positioned on opposite sides of the fold 118. In accordance with this embodiment, the buttresses 114 are annular shaped with a central aperture 140 through which a t-tag fastener 112 may be inserted in the manner described herein. Once the buttresses 114 are held against the tissue, a t-tag fastener 112 is applied through the central aperture 140 of the buttresses 114 in a manner holding the fold 118 together and securing the internal buttress 114 along opposite sides of the fold 118.

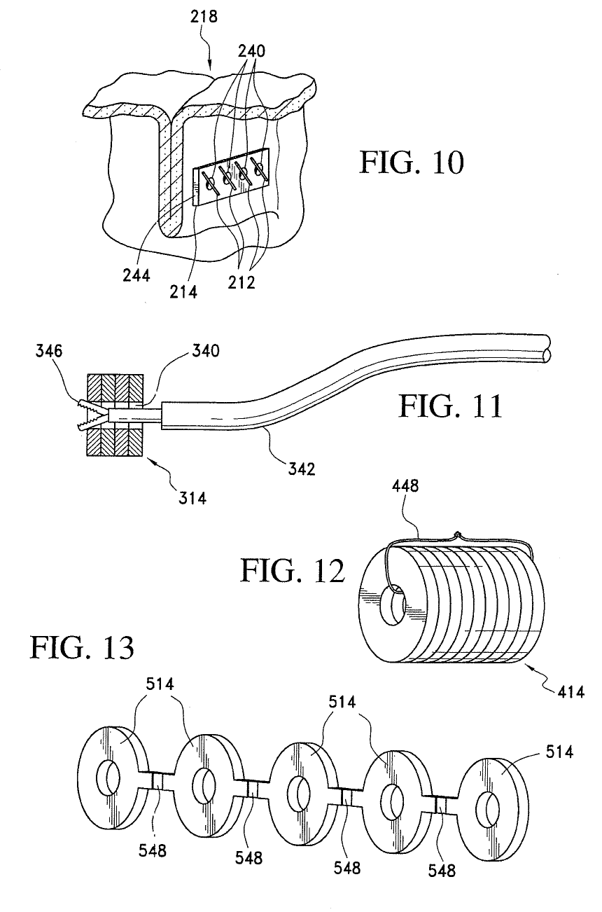

In accordance with an alternate embodiment, and with reference to FIG. 10, an apparatus and method for serosa-to-serosa fold 218 construction is shown. In accordance with this embodiment, the internal buttress 214 is an elongated member 244 including a plurality of apertures 240 (that is, an elongated buttress). As such, and in accordance with this technique, a serosa-to-serosa fold 218 is first created and the internal buttresses 214 are then positioned along opposite sides of the fold 218. Thereafter, a plurality of t-tag fasteners 212 are respectively applied through the apertures 240 of the elongated buttresses 214 in a manner holding the fold 218 together and securing the internal elongated buttresses 214 along opposite sides of the serosa-to-serosa fold 218.

One difficulty in the implementation of such a procedure is the delivery of internal buttresses to the treatment site. This may be accomplished in a variety of manners as described herein.

In accordance with one mechanism, and with reference to FIG. 11, multiple internal buttresses 314 (for example, and in accordance with a preferred embodiment, annular washers as disclosed and described above) may be delivered at the same time. In particular, an endoscopic grasper 342 is inserted through the central aperture 340 of the internal buttresses 314. Several annular shaped buttresses 314 are inserted over the endoscopic grasper 342. The endoscopic grasper jaws 346 are opened to prevent the buttresses 314 from falling off the distal end of the endoscopic grasper 342. The endoscopic grasper 342 and buttresses 314 are then delivered trans-orally into the gastric cavity. The grasper jaws 346 of the endoscopic grasper 342 are closed and the endoscopic grasper 342 is retracted. The buttresses 314 fall off inside the gastric cavity where they can be retrieved when needed for reinforcement of the surgical site.

In accordance with an alternate embodiment, and with reference to FIG. 12, a series of buttresses 414 in the shape of annular washers are aligned along a longitudinal axis and a suture 448 is wrapped therearound to hold the buttresses 414 together. The grouped buttresses 414 may then be engaged by an endoscopic grasper which is delivered transorally into the gastric cavity and the buttresses 414 are released to fall off inside the gastric cavity 410 to be retrieved when needed for reinforcement of the surgical site.

In accordance with yet another embodiment, and with reference to FIG. 13, the buttresses 514 are formed with fracture zones (or lines) 548 therebetween. More particularly, a series of substantially annular buttresses 514, which are in the shape of annular washers when separated are connected via fracture zones 548. As such, the series of buttresses 514 forms an elongated member 550 which may subsequently be broken up into individual buttresses 514 when desired. In accordance with such an embodiment, the longitudinally extending series of buttresses 514 are engaged by an endoscopic grasper and delivered transorally into the gastric cavity. The endoscopic grasper is then released allowing the buttresses 514 to fall off into the gastric cavity where they are retrieved for later use. When retrieved, a user simply grasps the end buttress 514, manipulates it until the fracture line 548 between the adjacent buttress 514 is broken at which time the buttress 514 may be utilized at the surgical site in a desired manner.

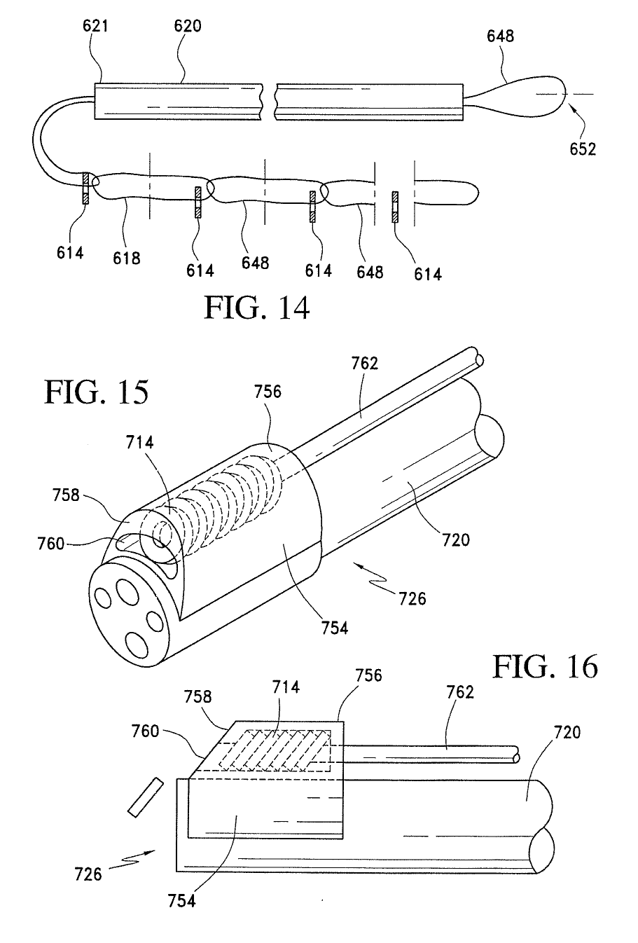

In contrast, and in accordance with an alternate embodiment shown with reference to FIG. 14, the buttresses 614, which are in the shape of annular washers, may be delivered one at a time. In accordance with this embodiment, the buttresses 614 are delivered each with a loop of suture 648. Each loop of suture 648 is tied to the next such that consecutive buttresses 614 are available as needed. The buttresses 614 are each pulled alongside the gastroscope 620 to the distal tip 621 of the gastroscope 620. To release each buttress 614 the suture loop 648 is cut at the handle 652 of the gastroscope 620, and one end of the suture loop 648 is pulled out, dropping off the buttress 614. This also leaves the next suture loop 648 ready to be picked up with the endoscopic graspers inside the gastric cavity. The cycle then repeats as needed until all buttress washers are delivered and used.

Referring to FIGS. 15 and 16, and in accordance with yet another embodiment for delivery of buttresses, a delivery device 726 is provided at the distal end of an gastroscope 720. The delivery device 726 includes a housing 754 in which a plurality of buttresses 714, which in accordance with a preferred embodiment are annular shaped washers, are stacked for subsequent dispensing. The housing 754 includes a proximal end 756 and a distal end 758. A dispensing aperture 760 is formed at the distal end 758 of the housing 754. The dispensing aperture 760 is spaced and dimensioned for selective release of the buttresses 714 held within the housing 754. Movement of the buttresses 714 toward the dispensing aperture 760 is achieved by the utilization of a push rod 762 that extends from the proximal end of the gastroscope 720 along the length of the gastroscope 720 and into the housing 754. The push rod 762 engages the buttresses 714 to force the stack of buttresses 714 toward the dispensing aperture 760 such that the buttresses 714 are dispensed from the dispensing aperture 760 in a controlled and efficient manner.

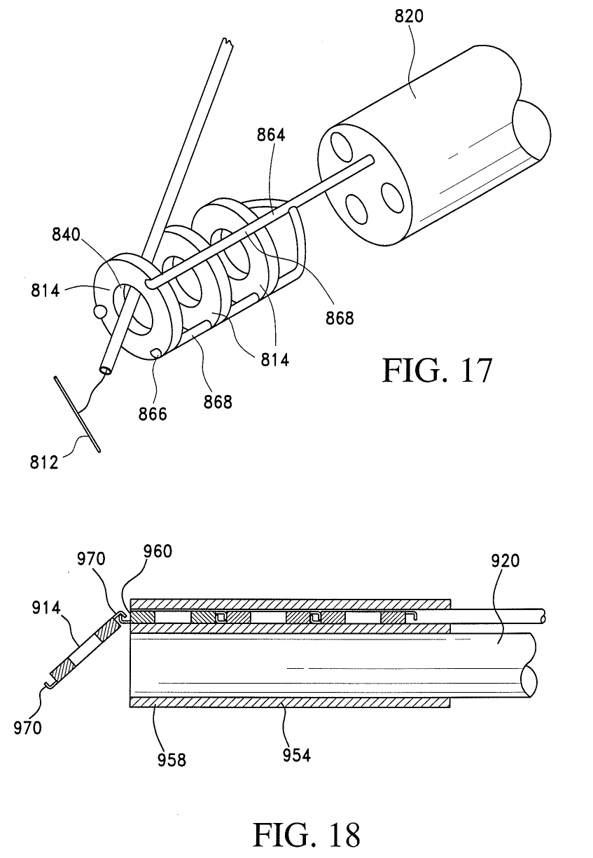

In accordance with yet another embodiment as shown in FIG. 17, the buttresses 814 are supported upon a rack 864 which extends through an gastroscope 820. At the distal end 866 of the rack 864, support members 868 are provided for selective engagement of a series of buttresses 814 which are aligned along a longitudinal axis such that the central apertures 840 thereof are in controlled alignment. As such, when one desires to withdraw a buttresses 814 for utilization in accordance with the present invention, a t-tag fastener 812 is forced through the central aperture 840 of a buttress 814 and then drawn towards the tissue. The force of drawing the buttress 814 toward the tissue removes it from the support rack 864 and allows the operator to pull the buttress 814 and t-tag fastener 812 toward the tissue for securement thereto.

In accordance with yet a further embodiment, and with reference to FIG. 18, the buttresses 914 may be longitudinally supported within a housing 954 at the distal end of the gastroscope 920. In accordance with this embodiment, the buttresses 914 include interlocking hooks 970 that allow them to be maintained in an aligned arrangement within the housing 954. The buttresses 914 may then be forced toward a dispensing aperture 960 at the distal end 958 of the housing 954 where a single buttress 914 is exposed and released from engagement with the adjacent buttresses 914. Because of the size of the housing 954 and the shape of the interlocking hooks 970, the buttresses 914 are retained in the interlocked configuration until such a time that the buttress exits the housing 954 at which time it is free to disengage from the adjacent buttress 914. The buttress may be constructed of a material or geometry that is conformable to the outer diameter of the endoscope so as to reduce the size of the circumscribed circle through which the device may pass.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used system is obtained and if necessary cleaned. The system can then be sterilized. In one sterilization technique, the system is placed in a closed and sealed container, such as a plastic or TYVEK bag. The container and system are then placed in a field of radiation that can penetrate the container, such as gamma radiation, x-rays, or high-energy electrons. The radiation kills bacteria on the system and in the container. The sterilized system can then be stored in the sterile container. The sealed container keeps the system sterile until it is opened in the medical facility.

It is preferred that the device is sterilized. This can be done by any number of ways known to those skilled in the art including beta or gamma radiation, ethylene oxide, and/or steam.

While the preferred embodiments have been shown and described, it will be understood that there is no intent to limit the invention by such disclosure, but rather, is intended to cover all modifications and alternate constructions falling within the spirit and scope of the invention.

1. A method for forming plications of a gastric cavity, comprising:
forming a tissue fold along a gastric wall of the gastric cavity;
securing the tissue fold with a fastener, wherein the step of securing includes positioning at least one buttress between tissue of the gastric wall and the fastener.

2. The method according to claim 1, wherein the fastener is a t-tag fastener.

3. The method according to claim 1, wherein the tissue fold is formed in an anterior wall of the gastric cavity.

4. The method according to claim 3, wherein the tissue fold is a serosa-to-serosa fold.

5. The method according to claim 1, wherein the at least one buttress is annular shaped.

6. The method according to claim 1, wherein the buttress is an elongated member including a plurality of apertures through which a plurality of fasteners are respectively applied in a manner holding the tissue fold together.

7. The method according to claim 1, further including the step of deploying multiple buttresses within the gastric cavity, wherein the step of deploying includes inserting a plurality of buttresses over an endoscopic grasper and opening grasper jaws of the endoscopic grasper to prevent the plurality of buttresses from falling off the endoscopic grasper, transorally delivering the endoscopic grasper to the gastric cavity and closing the grasper jaws of the endoscopic grasper to release the buttress within the gastric cavity.

8. The method according to claim 1, further including the step of deploying multiple buttresses within the gastric cavity, wherein the step of deploying includes aligning a series of buttresses along a longitudinal axis and wrapping a suture therearound to hold the buttresses together, engaging the buttresses with an endoscopic grasper, delivering the buttresses into the gastric cavity, and releasing the buttresses to fall off inside the gastric cavity.

9. The method according to claim 1, further including the step of deploying multiple buttresses within the gastric cavity, wherein the step of deploying includes providing a series of buttresses, which are connected via fracture zones allowing selective separation thereof, engaging the series of buttresses with an endoscopic grasper, delivering the buttresses transorally into the gastric cavity, manipulating the buttresses until the fracture zones between adjacent buttresses is broken at which time the buttress may be utilized at a surgical site in a desired manner.

10. The method according to claim 1, further including the step of deploying multiple buttresses within the gastric cavity, wherein the step of deploying includes delivering each buttress with a loop of suture, wherein each loop of suture is tied to a next loop of suture such that consecutive buttresses are available as needed, releasing individual buttresses by cutting the loop of suture releasing the buttress for use.

11. The method according to claim 1, further including the step of deploying multiple buttresses within the gastric cavity, wherein the step of deploying includes transorally positioning a delivery device within the gastric cavity, the delivery device includes a housing in which a plurality of buttresses are stacked for subsequent dispensing and a dispensing aperture for selective release of buttresses held within the housing, wherein movement of the buttresses toward the dispensing aperture is achieved by utilization of a push rod.

12. The method according to claim 1, further including the step of deploying multiple buttresses within the gastric cavity, wherein the step of deploying includes supporting a plurality of buttresses upon a rack, wherein the rack includes support members for selective engagement of a series of buttresses which are aligned along a longitudinal axis such that central apertures of the buttresses are in controlled alignment for release within the gastric cavity.

13. The method according to claim 1, further including the step of deploying multiple buttresses within the gastric cavity, wherein the step of deploying includes supporting the buttresses within a housing wherein the buttresses include interlocking hooks that allow them to be maintained in an aligned arrangement within the housing, forcing the buttresses toward a dispensing aperture of the housing where a single buttress is exposed and released from engagement with the adjacent buttresses.

14. The method according to claim 1, wherein the tissue fold is a serosa-to-serosa fold.
